(19) United States
(12) Patent Application Publication
(10) Pub. No.: US 2011/0125123 A1
(43) Pub. Date: May 26, 2011

(54) Paper Diaper with Attached Disposal Pouch

(76) Inventor: Goroh Moriji, Takashima-shi (JP)

(21) Appl. No.: 12/765,875

(22) Filed: Apr. 23, 2010

(30) Foreign Application Priority Data
Nov. 21, 2009 (JP) 2009-008324 U

(51) Int. Cl. A61F 13/15

(52) U.S. Cl. 604/385.13; 604/389

(57) ABSTRACT

Handy, aesthetically appealing paper diaper in which a disposal pouch is attached to part of the outer surface of the paper-diaper body. To all appearances the pouch cannot be seen, enabling hygienic disposal of the paper diaper without imparting a negative effect on its functionality. A disposal pouch, folded into a state in which its thickness is minimized, is interposed between a front sheet on the paper diaper and the outer surface of the paper-diaper body, or alternatively, between two plies composing a front-sheet section. An end part of the pouch is adhered to the body section in order to stop the pouch from coming loose when the paper diaper is worn, and a slit or a perforation is provided on the front-sheet section as a pouch-withdrawal opening, enabling the pouch to be withdrawn to let it be used when needed.

### BACKGROUND OF THE INVENTION

1. Technical Field

The present invention relates to a paper diaper with an attached disposal pouch, in which fitted to a paper diaper is a paper-diaper disposal pouch in order for the paper diaper to be contained and disposed of hygienically after use. It should be understood that “paper diapers” as employed in the present application include those in the form of briefs, and those of the type (adhesive-tape models) that when worn, the right and left sides are locked with fastening tape.

2. Description of the Related Art

For many years disposable paper diapers, beginning with baby-use, and diversifying into adult-use and pet-use, have been in widespread utilization in daily life.

From an early stage, numerous design innovations in paper diapers have been implemented, and in recent years paper diapers made of abundantly breathable and absorbent materials, and that are gentle on the skin and of superior comfort in use have arrived dominantly on the market, at comparatively low prices.

Attendant on the increase in demand for paper diapers of late, however, is the garbage problem—which is quite serious, especially as seen in environmental protection terms—with regard to the disposal of the paper diapers after use. It would be of great service to have available paper diapers with an attached disposal pouch that when worn are no different from conventional paper diapers, that are pleasant to use, and that enable convenient, hygienic disposal after use.

Therein, the present inventor conceived of a paper diaper with an attached disposal pouch that, compromising neither the breathability that is a key, primary function of a paper diaper, nor the mobility of the wearer, enables post-use disposal to be hygienic, without spoiling the charming appearance that pleases babies. That concept led to the creation of disposal-pouch-included paper diapers that provide both the functionality and aesthetic appeal of paper diapers, and the convenience of paper diapers when disposed of.
Patent Document 1: Japanese Unexamined Pat. App. Pub. No. 2007-236967.

### BRIEF SUMMARY OF THE INVENTION

An object of the present invention is to make available exceptionally convenient paper diapers with an attached disposal pouch that, without compromising either the functionality or the aesthetic appeal of conventional paper diapers, enables hygienic disposal of the paper diapers after use.

The present invention as given in a first aspect is a paper diaper with an attached disposal pouch, in which fitted to a paper diaper is a disposal pouch in order for the paper diaper after use to be hygienically disposed of, the paper diaper being furnished with: a paper-diaper body section having a front panel and a rear panel; fastening-tape tabs affixed to right- and left-side areas of the rear panel along the back thereof, and provided extending respectively rightward and leftward; a front-sheet section for holding the fastening tabs fast along their other ends not affixed to the rear panel; and a disposal pouch; wherein, to impart both convenience and aesthetic appeal: said front-sheet section along a peripheral portion of its reverse side has an adhesive surface, on a midportion of the reverse side has a non-adhesive region not possessing an adhesive surface, and is adhered to the paper-diaper body front-panel outer surface along the abdomen; said disposal pouch, of a thin material in order to minimize its thickness, is arranged in a folded state in a gap between the paper-diaper body outer surface and the non-adhesive region of the reverse side of said front-sheet section; said disposal pouch is made of an impermeable sheet material so as to enable sealed enclosure of the paper diaper after use to confine the foul odors of waste to a minimum during disposal; said disposal pouch is arranged in such a way as not to come loose from the paper diaper easily; and in at least one location on said front sheet an opening is provided for withdrawal of said disposal pouch stored inside.

The present invention as given in a second aspect is a paper diaper with an attached disposal pouch, in which fitted to a paper diaper is a disposal pouch in order for the paper diaper after use to be hygienically disposed of, the paper diaper being furnished with: a paper-diaper body section having a front panel and a rear panel; fastening-tape tabs affixed to right- and left-side areas of the rear panel along the back thereof, and provided extending respectively rightward and leftward; a front-sheet section for holding the fastening tabs fast along their other ends not affixed to the rear panel; and a disposal pouch; wherein, to impart both convenience and aesthetic appeal: said front-sheet section is arranged layered in two sheets on the outer surface of the paper-diaper body section, with the underlying front sheet being adhered to the paper-diaper body front-panel outer surface along the abdomen, and with the overlying front sheet along a peripheral portion of its reverse side having an adhesive surface, on a midportion of its reverse side having a non-adhesive region not possessing an adhesive surface, and being adhered onto, so as to overlie, the underlying front sheet; said disposal pouch, of a thin material in order to minimize its thickness, is arranged in a folded state in a gap between the underlying front sheet and the non-adhesive region of the reverse side of the overlying front sheet; said disposal pouch is made of an impermeable sheet material so as to enable sealed enclosure of the paper diaper after use to confine the foul odors of waste to a minimum during disposal; said disposal pouch is arranged in such a way as not to come loose from the paper diaper easily; and in at least one location on said overlying front sheet an opening is provided for withdrawal of said disposal pouch stored inside.

The present invention as given in a third aspect involves a disposal-pouch-included paper diaper as given in either the first or second aspects, wherein said disposal pouch is characterized in that part of the pouch is adhered either to the diaper body, or to said front sheet.

The present invention as given in a fourth aspect involves a disposal-pouch-included paper diaper as given in any one of the first through third aspects, characterized in that: a perforation of a length that lets the contained disposal pouch be withdrawn is provided for the disposal-pouch withdrawal opening; and the perforation is located along at least one of the longer edges, the shorter edges, or the midsection of the rectangular front sheet.

The present invention as given in a fifth aspect involves a disposal-pouch-included paper diaper as given in any one of the first through third aspects, characterized in that: a slit of a length that lets the contained disposal pouch be withdrawn is provided for the disposal-pouch withdrawal opening; the disposal-pouch withdrawal opening is located along at least one of the longer edges or the shorter edges of the rectangular front sheet; and part of the slit for the disposal-pouch withdrawal opening is locked by sticky tape to prevent the disposal pouch from coming loose.

The present invention as given in a sixth aspect involves a disposal-pouch-included paper diaper as given in any one of the first through third aspects, characterized in that: a slit of a length that lets the folded and contained disposal pouch be withdrawn is provided for the disposal-pouch withdrawal opening; the disposal-pouch withdrawal opening is located along at least one of the longer edges or the shorter edges of the rectangular front sheet; and the slit for the pouch withdrawal opening is fitted with openable/closable Velcro™ to prevent the disposal pouch from coming loose.

The present invention as given in a seventh aspect is a paper diaper with an attached disposal pouch, in which fitted to a paper diaper is a disposal pouch in order for the paper diaper after use to be hygienically disposed of, the paper diaper being furnished with: a paper-diaper body section having a front panel and a rear panel; a front-sheet section; and a disposal pouch; wherein, to impart both convenience and aesthetic appeal: said front-sheet section along a peripheral portion of its reverse side has an adhesive surface, on a midportion of its reverse side has a non-adhesive region not possessing an adhesive surface, and is adhered to a waist-portion outer surface of the paper diaper body; said disposal pouch, of a thin material in order to minimize its thickness, is arranged in a folded state in a gap between the paper-diaper body outer surface and the non-adhesive region of the reverse side of said front-sheet section; said disposal pouch is made of an impermeable sheet material so as to enable sealed enclosure of the paper diaper after use to confine the foul odors of waste to a minimum during disposal; said disposal pouch is arranged in such a way as not to come loose from the paper diaper easily; and in at least one location on said front sheet an opening is provided for withdrawal of said disposal pouch stored inside.

The present invention as given in an eighth aspect is a paper diaper with an attached disposal pouch, in which fitted to a paper diaper is a disposal pouch in order for the paper diaper after use to be hygienically disposed of, the paper diaper being furnished with: a paper-diaper body section having a front panel and a rear panel; a front-sheet section; and a disposal pouch; wherein, to impart both convenience and aesthetic appeal: said front-sheet section is arranged layered in two plies on a waist-portion outer surface of the paper-diaper body section, with the underlying front sheet being adhered to the paper-diaper body outer surface, and with the overlying front sheet along a peripheral portion of its reverse side having an adhesive surface, on a midportion of its reverse side having a non-adhesive region not possessing an adhesive surface, and being adhered onto, so as to overlie, the underlying front sheet; said disposal pouch, of a thin material in order to minimize its thickness, is arranged in a folded state in a gap between the underlying front sheet and the non-adhesive region of the reverse side of the overlying front sheet; said disposal pouch is made of an impermeable sheet material so as to enable sealed enclosure of the paper diaper after use to confine the foul odors of waste to a minimum during disposal; said disposal pouch is arranged in such a way as not to come loose from the paper diaper easily; and in at least one location on said overlying front sheet an opening is provided for withdrawal of said disposal pouch stored inside.

The present invention as given in a ninth aspect involves a disposal-pouch-included paper diaper as given in either the seventh or eighth aspects, wherein said disposal pouch is characterized in that part of the pouch is adhered either to the diaper body, or to said front sheet.

The present invention as given in a tenth aspect involves a disposal-pouch-included paper diaper as given in any one of the seventh through ninth aspects, characterized in that: a slit of a length that lets the folded and contained disposal pouch be withdrawn is provided for the disposal-pouch withdrawal opening; the disposal-pouch withdrawal opening is located along at least one of the longer edges or the shorter edges of the rectangular front sheet; and part of the disposal-pouch withdrawal opening is locked by sticky tape.

The present invention as given in a eleventh aspect involves a disposal-pouch-included paper diaper as given in any one of the seventh through ninth aspects, characterized in that: a perforation of a length that lets the folded and contained disposal pouch be withdrawn is provided for the disposal-pouch withdrawal opening; and the perforation is located along at least one of the longer edges, the shorter edges, or the midsection of the rectangular front sheet.

The present invention as given in a twelfth aspect involves a disposal-pouch-included paper diaper as given in any one of the seventh through ninth aspects, characterized in that: a slit of a length that lets the folded and contained disposal pouch be withdrawn is provided for the disposal-pouch withdrawal opening; the disposal-pouch withdrawal opening is located along at least one of the longer edges or the shorter edges of the rectangular front sheet; and the slit for the pouch withdrawal opening is fitted with openable/closable Velcro™ to prevent the disposal pouch from coming loose.

The present invention as given in a thirteenth aspect involves a disposal-pouch-included paper diaper as given in any one of the first through twelfth aspects, characterized in that: a perforation is provided in said paper-diaper disposal pouch in the vicinity of the end thereof adhered to the diaper body or the front sheet, whereby said disposal pouch may be used by tearing the pouch off by the perforation and taking it out.

In accordance with a disposal-pouch-included paper diaper according to the present invention, during paper-diaper disposal, a post-use paper diaper is rolled up, the disposal pouch is pulled out through the withdrawal opening in the front sheet arranged on the paper diaper body, and after the paper diaper is put in, the entrance of the pouch is bound, thereby preventing bad smells and making it possible to dispose of the paper diaper simply and hygienically. Additional benefits that will be appreciated of a disposal-pouch-included paper diaper according to the present invention are that the traditional appearance of paper diapers is not marred, nor is their functionality such as breathability negatively influenced. Furthermore, the inexpensiveness in that what is added to the traditional product is chiefly a disposal pouch holds down manufacturing cost increases to a minimum.

### BRIEF DESCRIPTION OF THE SEVERAL VIEWS OF THE DRAWINGS

FIG. 1 is a simplified explanatory elevational diagram representing the constitution, involving a first mode of embodying the present invention, of a paper diaper in the form of briefs with an attached disposal pouch.

FIG. 2 is a perspective view representing a post-use disposal-pouch-included paper diaper of the present invention, having been rolled up.

FIG. 3 is a perspective view representing a disposal pouch having been pulled out from a disposal-pouch-included paper diaper of the present invention.

FIG. 4 is a perspective view representing one example of an instance in which a used paper diaper has been put inside a drawn-out disposal pouch.

FIG. 5 is an explanatory elevational view serving to illustrate a disposal-pouch insert having been withdrawn through a pouch-withdrawal opening.

FIG. 6 is a simplified explanatory elevational diagram representing the constitution, involving a second mode of embodying the present invention, of a paper diaper of the adhesive-tape type with an attached disposal pouch.

FIG. 7 is a perspective view representing a disposal pouch having been pulled out from a disposal-pouch-included paper diaper involving the second mode of embodying the present invention, with the paper diaper having been put into a rolled-up state.

### DETAILED DESCRIPTION OF THE INVENTION

Modes of embodying the invention involve a disposal-pouch-included paper diaper in which a pouch for disposal is contained, folded in a state in which its outward appearance is indiscernible, in between a front sheet arranged along the front-panel abdomen of paper diapers of the adhesive-tape type or paper diapers in the form of briefs (the front sheet being a striplike sheet material provided in order for fastening tabs provided on the right and left of the rear panel of paper diapers of the adhesive-tape type to be adhered detachably/reattachably to the front panel, wherein sheets patterned with cute designs so as to appeal to infants and toddlers are common), and the outer surface of the paper-diaper body, and when needed can be withdrawn to enable hygienic disposal of the paper diaper, while maintaining the exceptional functionality of conventional paper diapers. It should be noted that the position of the front sheet should be in a single location on the outer surface of the paper-diaper body along the waist, and is not limited to being along the abdomen. Likewise, it should be understood that while paper diapers of the present invention are discussed with infants/toddlers in mind, the diapers may be adult-use or pet-use.

A feature of a paper diaper of the present invention is that with a disposal pouch being attached to the paper diaper, when the paper diaper is to be discarded, the pouch may be readily withdrawn to allow the paper diaper to be conveniently and hygienically disposed of, while the invention is also characterized in that the disposal pouch, by being hidden inside the front sheet where it cannot be seen, does not mar the design character of the paper diaper overall, and also does not exert any negative influence on such vital characteristics of paper diapers as breathability, ease of putting on/taking off, and comfort of fit.

### Embodiment 1

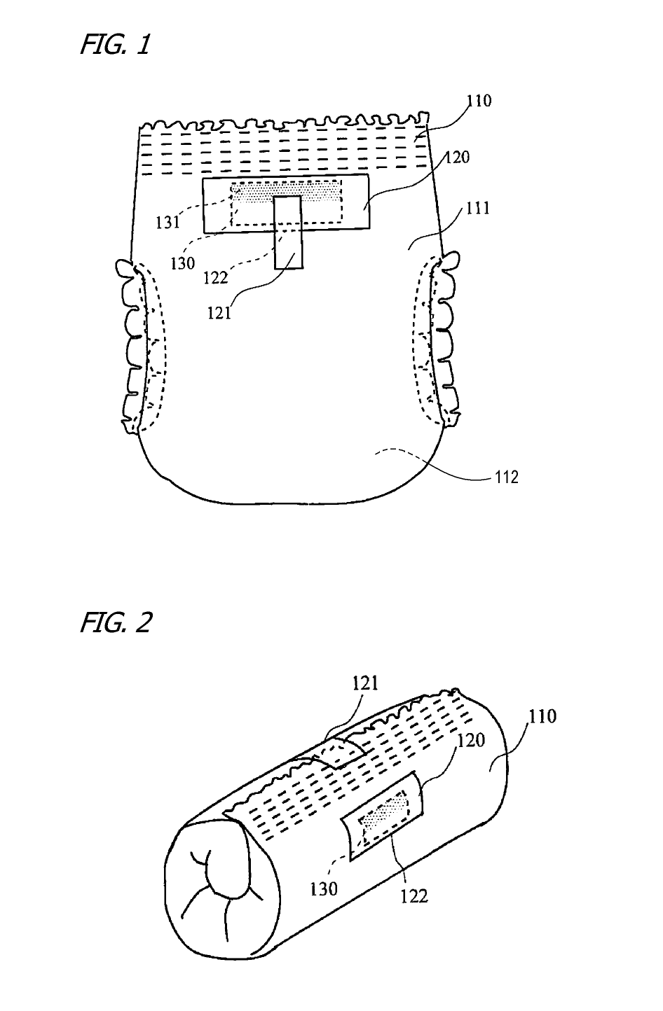

FIG. 1 is a simplified explanatory elevational diagram representing the constitution, involving a first mode of embodying the present invention, of a paper diaper in the form of briefs with an attached disposal pouch.

A disposal-pouch-included paper diaper involving the present invention represented in FIG. 1 is a paper diaper in the form of briefs, which is the type that is put on like underpants, and includes a paper-diaper body section having a front panel and a rear panel, a front-sheet section, a disposal-pouch section contained in a gap between the paper-diaper body section outer surface and the front-sheet section, and a sticky-tape section. In FIG. 1 the front-sheet section is arranged on the rear panel of the paper-diaper body section. Nevertheless, the position of the front-sheet section is not thereby limited, and may be in the middle or on either the right- or left-side areas of the front panel of the paper-diaper body section, with a location that does not negatively affect the functionality of the paper diaper being preferable.

In FIG. 1, reference numeral 110 denotes the paper-diaper body section, reference numeral 111 denotes the rear panel of the paper-diaper body section, and reference numeral 112 denotes the front panel of the paper-diaper body section (reference numeral 112 corresponds to the reverse side of what is denoted by numeral 111 and thus its lead line is partially in phantom in the figure). Reference numeral 120 denotes the front-sheet section, which is disposed on the outer surface of the paper-diaper body-section along the back of the rear panel 111. Reference numeral 130 denotes the paper-diaper disposal pouch, which is housed, in a folded state to minimize the thickness of the pouch, in a gap between the front-sheet section 120 and the outer surface of the body section 110 (in the FIG. 1 case, the rear panel 111), wherein as long as it has not been pulled out, the pouch cannot be seen from without. Reference numeral 131 denotes part of the disposal pouch 130, which is a portion that is adhered to the body section or the reverse side of the front sheet so that the pouch does not come loose.

In the case of ordinary paper diapers of the adhesive-tape type, the front sheet is arranged by adhering the entire reverse side of the front sheet to the outer surface of the front panel of the paper-diaper body, but in the case of a front sheet of the present invention, only the peripheral portion of the front-sheet reverse side is adhered to the paper-diaper body outer surface, with the portion of the reverse side other than the periphery (the front-sheet midportion) not being adhered, whereby a cavity is formed. Further, in FIG. 1, on part of the lower of the longer edges of the front sheet, as a withdrawal opening to allow the pouch to be pulled out, the sheet has a non-adhered portion (122) in part of the peripheral portion of the reverse side, enabling the pouch inside to be pulled out through this slit 122. Thus, the disposal pouch can be housed in a tiny, thin, folded state in the cavity portion along the inner side of the front sheet, and at the same time can be pulled out freely through the withdrawal opening 122.

The disposal pouch 130 is made of an impermeable sheet material so as to enable sealed enclosure to confine the foul odors of waste to a minimum, and being of a thin material in order to minimize its thickness is arranged in a folded state in a gap between the front sheet 120 and the paper-diaper body 110 outer surface; furthermore, so as not to come loose from the paper diaper easily even though the infant/toddler wearing the paper diaper is moving about, part (131) of the disposal pouch is adhered/fixed to the diaper-body outer surface or the front-sheet reverse side.

Reference numeral 122 denotes a withdrawal opening to allow the disposal pouch to be pulled out, and is provided on part of the front-sheet section 120. In FIG. 1, the withdrawal opening is provided on part of the lower of the front-sheet 120 longer edges, and because it overlaps with the longer edge of the front sheet the withdrawal opening cannot be seen. Reference numeral 121 denotes sticky tape. In the case of disposal-pouch tape arranged on an ordinary paper diaper in the form of briefs, when the paper diaper is to be discarded, the tape is for retaining the used paper diaper in a rolled-up state, and is arranged folded on the body-section outer surface. The sticky tape 121 in FIG. 1 is arranged on the outer surface of the front sheet over the withdrawal opening 122, and serves in the role of keeping the disposal pouch contained inside from sticking out of or coming loose from the withdrawal opening 122 in the front-sheet section.

It should be understood that in the present invention, in order to keep the disposal pouch from popping out, the folded sticky tape for discarding (disposal tape) may be employed, but is not mandatory; it may be sticky tape shorter than the length of the disposal tape. Likewise, sticky tape is not an absolute necessity; it is also possible by other methods to keep the disposal pouch from popping out.

The position where the front sheet is arranged should be a location that, on account of the front sheet having been put in place, is not apt to adversely affect the nature of the infant's/toddler's movements, or the breathability of the paper diaper, but is not limited to being along the body rear panel as in FIG. 1. Furthermore, although not depicted in FIG. 1, in the same way as with ordinary paper diapers, the front sheet for a paper diaper of the present invention is patterned with an infant-pleasing design so as to contribute to the decorative feel of the paper diaper.

With the front-sheet section 120 in FIG. 1 being single-ply, between it and the body-section outer surface 111 the disposal pouch 130 is contained, but as an alternative configuration the front sheet may be two-ply. In that case, onto an underlying of the front sheets, adhered/fixed to the paper-diaper body outer surface, a front sheet of the same configuration is overlaid, and the peripheral portion of the reverse side of the overlying front sheet is adhered to the outer surface of the underlying front sheet, and by providing as a disposal-pouch withdrawal opening an area where an adhesive surface is not created, on part of the reverse-side peripheral adhesive surface, the disposal pouch can be contained between the two upper/lower front sheets so that the pouch can be pulled out when needed. As far as the other features in the implementation having the front sheet be two-ply are concerned, they are the same as with the single-ply implementation.

FIG. 2, involving the first mode of embodying the present invention as represented in FIG. 1, is a perspective view representing a disposal-pouch-included paper diaper having been rolled up in order to be discarded. In implementations in which disposal tape 121 is fastened to the paper diaper, it is employed so that the used paper diaper 110 may be retained in a tightly rolled-up state. Employing the disposal tape to retain the paper diaper in a rolled-up state is not mandatory, however, and in implementations in with no disposal tape, the paper diaper may be doubled over and then put into the disposal pouch.

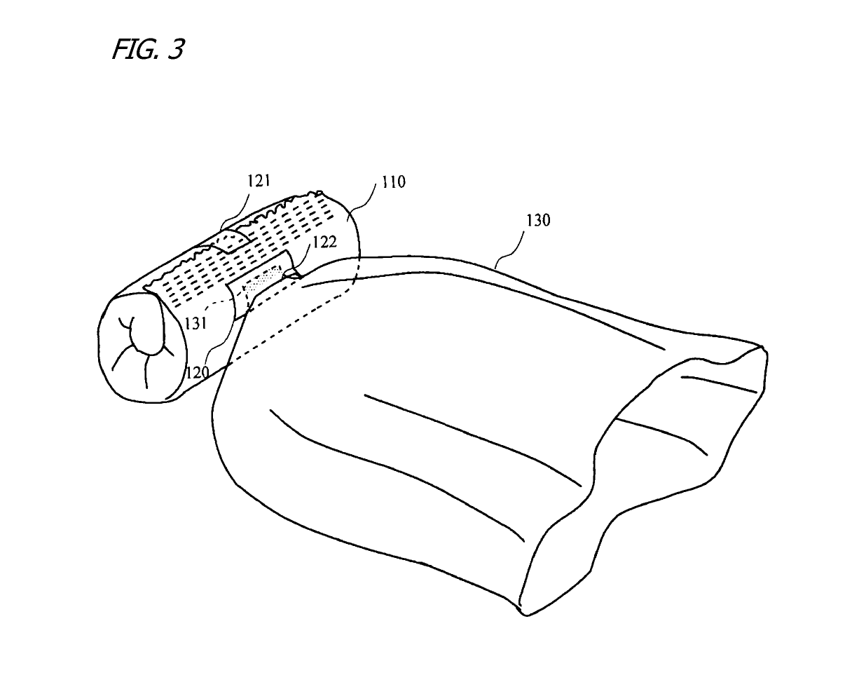

FIG. 3 is a perspective view representing a situation in which after a disposal-pouch-included paper diaper of the present invention, as represented in FIG. 2, has been rolled up tightly in order to be discarded after use, the disposal pouch 130 has been withdrawn from behind/within the front sheet(s) 120, through the withdrawal opening 122. One end of the disposal pouch 130 is adhered (131) to the body-section outer surface or the front-sheet reverse side, to anchor the pouch when it is pulled out.

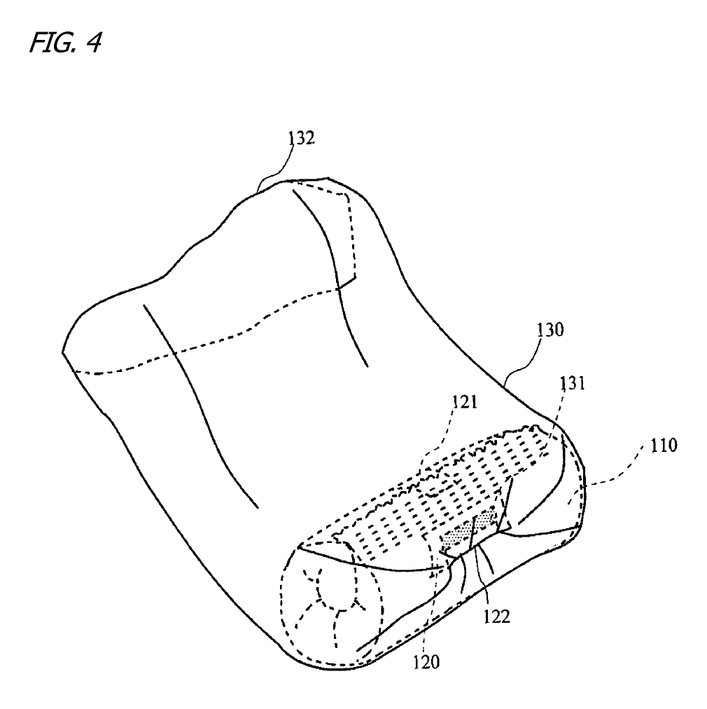

FIG. 4 is a perspective view representing a situation in which a disposal-pouch-included paper diaper of the present invention after having been rolled up tightly, as represented in FIG. 3, in order to be discarded after use has been put inside the disposal pouch 130 after the disposal pouch 130 has been withdrawn from behind/within the front sheet(s) 120, through the withdrawal opening 122. The pulled-out disposal pouch 130 is turned inside-out, and after the paper diaper has been put inside, the mouth of the disposal pouch 130 is knotted to enable the pouch to be hygienically discarded without letting bad smells escape. It should be noted that the disposal pouch is manufactured of a material enabling it to be discarded together with the paper diaper as combustible garbage. And it should be understood that FIG. 4 is a single example; depending on the location where the disposal-pouch withdrawal opening is arranged, the manner of insertion into the pouch will differ.

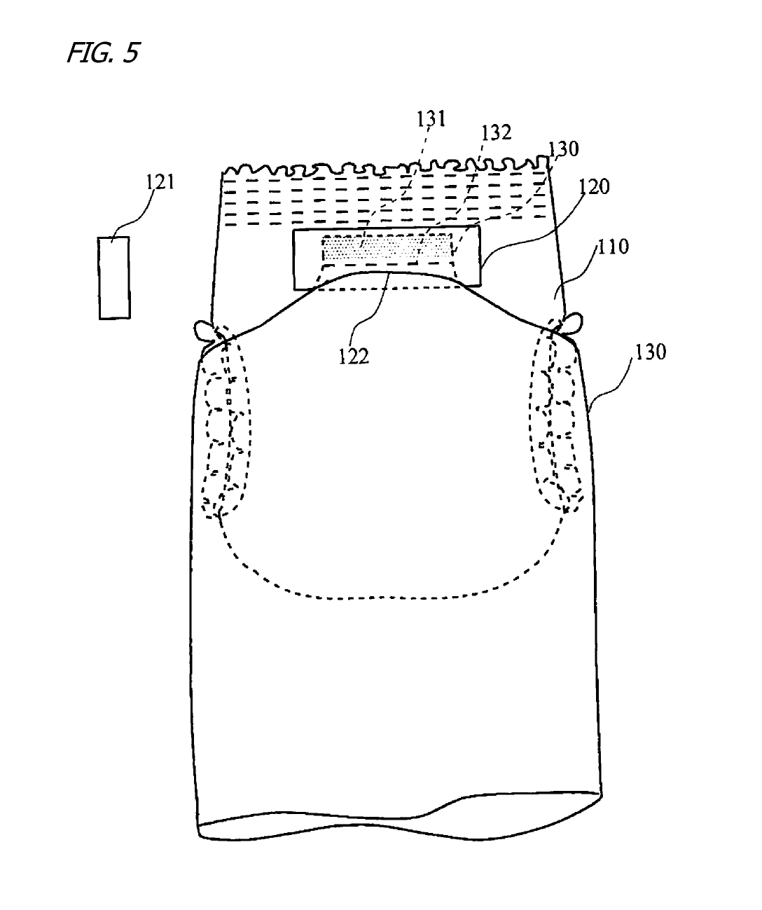

FIG. 5 is an explanatory elevational view serving to illustrate a situation in which a disposal-pouch insert has been withdrawn through a pouch-withdrawal opening in a front sheet. Reference numeral 122 denotes the pouch-withdrawal opening, while reference numeral 130 denotes the disposal pouch having been pulled out. Reference numeral 132 denotes a perforation provided in proximity to a disposal-pouch adherence surface 131. Reference numeral 121 denotes sticky tape, and while usually it impermanently locks the disposal-pouch withdrawal opening 122, it has been removed in order that the pouch 130 may be withdrawn.

The adherence surface 131 provided on part of the disposal pouch is adhered to the paper-diaper body or part of the front-sheet reverse side, where it prevents the pouch from coming loose when the paper diaper is worn. When the paper diaper is to be discarded after use, the disposal pouch 130 is torn off along the perforation 132 where the pouch is provided in the proximity of 131, as indicated in FIG. 5, and after the pouch has been pulled apart from the paper diaper, the paper diaper is put into the pouch so that it can be thrown away.

### Embodiment 2

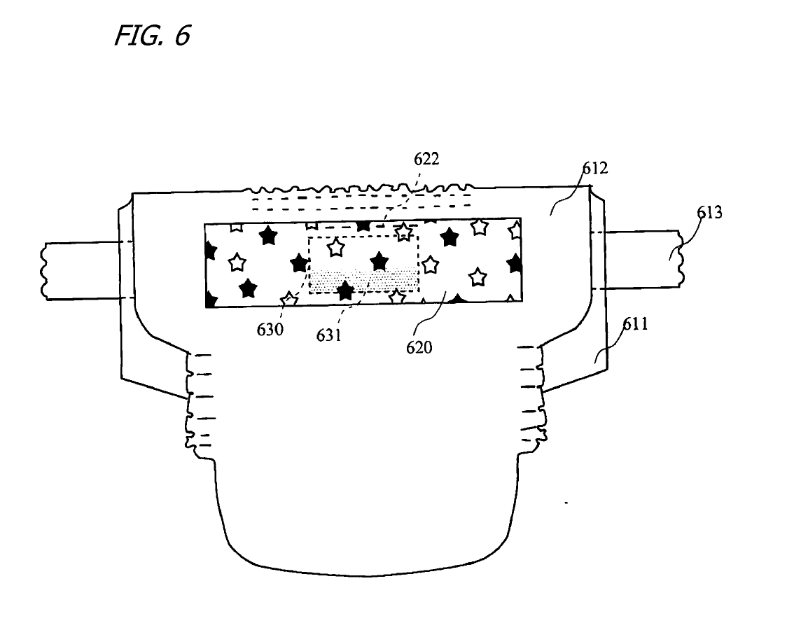

FIG. 6 is a simplified explanatory elevational diagram representing the constitution, involving a second mode of embodying the present invention, of a paper diaper of the adhesive-tape type with an attached disposal pouch.

A disposal-pouch-included paper diaper involving the present invention as represented in FIG. 6 is an adhesive-tape model of paper diaper, being a type that is worn by detachably/reattachably adhering, to a front sheet provided on the front panel of the paper diaper, fastening tabs provided on the right and left of the rear panel, and includes a paper-diaper body section having a front panel and a rear panel, fastening-tape tabs provided on the right- and left-side areas of the rear panel, a front-sheet section, and a disposal-pouch section contained in a gap between the front-sheet section and the outer surface of the paper-diaper body section.

In the case of ordinary paper diapers for infants/toddlers, cute patterns of animals, flowers or other images designed to please infants and toddlers are printed onto the front-sheet section. Inasmuch as a disposal pouch of the present invention is folded up and arranged along the inner side of the front sheet, and in the state in which the pouch has not been pulled out is invisible in terms of the outward appearance of the diaper, the appealing nature of the design on the front sheet will not in any way be spoiled.

Also, with the adhesive-tape model of paper diaper in FIG. 6, the front sheet for holding the fastening tabs fast is arranged on the outer surface of the paper-diaper body section along the abdomen, with the disposal pouch being contained thereunder, in a state in which pouch has been folded, but the location of the front sheet containing the disposal pouch is not limited to along the body-section abdomen; it is possible to arrange the sheet on the body waist-portion outer surface along the sides, or on the waist-portion outer surface along the back.

In FIG. 6, reference numeral 610 denotes the paper-diaper body section, reference numeral 611 denotes the back panel of the paper-diaper body section, and reference numeral 612 denotes the front panel of the paper-diaper body section. Reference numeral 620 denotes the front-sheet section, which is disposed on the outer surface of the paper-diaper body-section front panel 612 along the abdomen. Reference numeral 630 denotes the paper-diaper disposal pouch, which is housed behind the front-sheet section 620 and cannot be seen from without. Reference numeral 613 denotes fastening-tape tabs, which are respectively affixed to right- and left-side areas of the paper-diaper rear panel 611 along the back thereof, and are provided extending rightward and leftward; wherein when the paper diaper is worn, the other ends of the fastening tabs 613 can each be detachably/reattachably affixed to the front-sheet section 620 arranged on the outer surface of the front panel (612) along the abdomen. It will be appreciated that while a star design is patterned on the front sheet 620 in FIG. 6, the pattern may be of animals, flowers, etc., in that the character of the design on the paper diapers that come with decorations to appeal to infants/toddlers is not impeded by the attaching of a disposal pouch of the present invention. Reference numeral 622 denotes a perforation provided as a disposal-pouch withdrawal opening. Reference numeral 631 denotes a portion of the disposal pouch that is adhered to the paper-diaper body outer surface or the front-sheet reverse side in order to prevent the pouch from coming loose.

Likewise as with the first embodying mode, the method whereby the front sheet is adhered in the present mode of embodying the invention is that not the entire reverse side of the front sheet is adhered to the outer surface of the paper-diaper body; rather, the adhesive surface is only the portion of the front-sheet reverse side on the periphery, such that the portion of the reverse side other than the periphery (the front-sheet midportion) is not adhered, making a cavity. The disposal pouch can be contained in a folded-up state in the cavity.

Further, in FIG. 6, a perforation is provided as a disposal-pouch withdrawal opening, which makes it possible to break the perforation when needed, to allow the pouch insert to be taken out. In the FIG. 6 instance, the perforation is provided in the vicinity of the upper of the front-sheet's longer edges, but the position may be a location that is along any one of the lower of the longer edges, the shorter edges or the midportion.

The disposal-pouch withdrawal opening may also be other than a perforation; it may be a type in which, as with the withdrawal opening 122 in FIG. 1, in part of the periphery of the front sheet, in the reverse side, a portion not having an adhesive surface is provided, and through such slit the pouch insert is pulled out.

The disposal pouch 630, of a thin material in order to minimize its thickness, is arranged in a folded state in a gap between the front-sheet section 620 and the paper-diaper body outer surface 612, wherein when the paper diaper is worn, as long as the disposal pouch 630 is not pulled out, it is hidden to all appearances and cannot be seen. Further, with the disposal pouch 630, so as not to come loose from the paper diaper easily when an infant or toddler is wearing the paper diaper, despite the infant's/toddler's moving about, part of the pouch is arranged adhered to the diaper body or the front sheet, in which respect the pouch is similar to that of Embodiment 1 represented in FIG. 1.

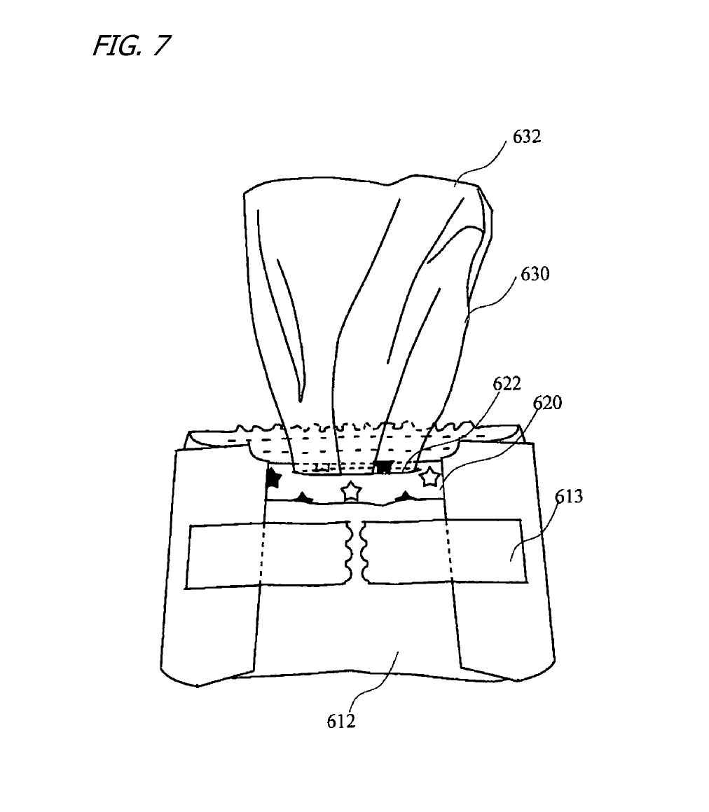

FIG. 7 is a perspective view representing a state in which in order to discard a disposal-pouch-included paper diaper after use, the disposal pouch has just been pulled out from the paper diaper, which has been put into a tightly rolled-up state. As for the disposal pouch 630, the perforation 622 provided in the vicinity of the upper of the longer edges of the front sheet 620 is broken and the disposal pouch 630 is withdrawn from inside. The post-use paper diaper having been rolled up tightly is put into the pulled-out disposal pouch 630 and the entrance 632 of the pouch is bound, thereby enabling hygienic disposal of the pouch.

The disposal pouch 630 may be of a type, as represented in FIG. 6, in which a perforation is provided in the vicinity of the pouch's adhered portion 631, wherein when the paper diaper is to be discarded the pouch is torn off along the perforation to separate it from the paper diaper for use.

In FIGS. 6 and 7, the front sheet 620 is single-ply, but the constitution may be, as described in Embodiment 1, one in which the front sheet is arranged by overlaying two laminae and the disposal pouch is contained in between the two front-sheet laminae. In respect of the disposal-pouch-included paper diaper involving Embodiment 2, although with the paper-diaper type being an adhesive-tape model, dissimilarities arise owing to the fact that it differs from the briefs model of Embodiment 1, it is otherwise the same as that of Embodiment 1 and therefore the description of the remaining details is omitted.

Also, the paper diapers of Embodiments 1 and 2 of the present invention are characterized by a disposal pouch being attached to a publicly known paper diaper, and because the method of creating the paper-diaper body is publicly known, its detailed description is omitted.

In accordance with the present invention, containing a disposal pouch on part of the outer surface of a paper-diaper body, in a manner in which it cannot be seen from without and in a state in which its thickness is minimized, makes available a paper diaper that, by withdrawal of the pouch when needed, affords convenience and at the same time excels in aesthetic appearance.

What is claimed is:

1. A paper diaper with an attached disposal pouch, in which fitted to a paper diaper is a disposal pouch in order for the paper diaper after use to be hygienically disposed of, the paper diaper comprising:
a paper-diaper body section having a front panel and a rear panel;
fastening-tape tabs affixed to right- and left-side areas of the rear panel along the back thereof, and provided extending respectively rightward and leftward;
a front-sheet section for holding the fastening tabs fast along their other ends not affixed to the rear panel; and
a disposal pouch; the disposal-pouch-included paper diaper characterized in that to impart both convenience and aesthetic appeal:
said front-sheet section along a peripheral portion of its reverse side has an adhesive surface, on a midportion of the reverse side has a non-adhesive region not possessing an adhesive surface, and is adhered to the paper-diaper body front-panel outer surface along the abdomen;
said disposal pouch, of a thin material in order to minimize its thickness, is arranged in a folded state in a gap between the paper-diaper body outer surface and the non-adhesive region of the reverse side of said front-sheet section;
said disposal pouch is made of an impermeable sheet material so as to enable sealed enclosure of the paper diaper after use to confine the foul odors of waste to a minimum during disposal;
said disposal pouch is arranged in such a way as not to come loose from the paper diaper easily; and
in at least one location on said front sheet an opening is provided for withdrawal of said disposal pouch stored inside.

2. A disposal-pouch-included paper diaper as set forth in claim 1, the disposal-pouch-included paper diaper being wherein said disposal pouch is characterized in that part of the pouch is adhered either to the diaper body, or to said front sheet.

3. A disposal-pouch-included paper diaper as set forth in claim 1, the disposal-pouch-included paper diaper characterized in that:
a perforation of a length that lets the contained disposal pouch be withdrawn is provided for the disposal-pouch withdrawal opening;
the front sheet is rectangular in form; and
the perforation is located along at least one of the longer edges, the shorter edges, or the midsection of the rectangular front sheet.

4. A disposal-pouch-included paper diaper as set forth in claim 1, the disposal-pouch-included paper diaper characterized in that:
a slit of a length that lets the contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
part of the slit for the disposal-pouch withdrawal opening is locked by sticky tape to prevent the disposal pouch from coming loose.

5. A disposal-pouch-included paper diaper as set forth in claim 1, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
the slit for the pouch withdrawal opening is fitted with openable/closable Velcro™ to prevent the disposal pouch from coming loose.

6. A disposal-pouch-included paper diaper as set forth in claim 1, the disposal-pouch-included paper diaper characterized in that:
a perforation is provided in said paper-diaper disposal pouch in the vicinity of the end thereof adhered to the diaper body or the front sheet, whereby
said disposal pouch may be used by tearing the pouch off by the perforation and taking it out.

7. A paper diaper with an attached disposal pouch, in which fitted to a paper diaper is a disposal pouch in order for the paper diaper after use to be hygienically disposed of, the paper diaper comprising:
a paper-diaper body section having a front panel and a rear panel;
fastening-tape tabs affixed to right- and left-side areas of the rear panel along the back thereof, and provided extending respectively rightward and leftward;
a front-sheet section for holding the fastening tabs fast along their other ends not affixed to the rear panel; and
a disposal pouch; the disposal-pouch-included paper diaper characterized in that to impart both convenience and aesthetic appeal:
said front-sheet section is arranged layered in two sheets on the outer surface of the paper-diaper body section, with the underlying front sheet being adhered to the paper-diaper body front-panel outer surface along the abdomen, and with the overlying front sheet along a peripheral portion of its reverse side having an adhesive surface, on a midportion of its reverse side having a non-adhesive region not possessing an adhesive surface, and being adhered onto, so as to overlie, the underlying front sheet;
said disposal pouch, of a thin material in order to minimize its thickness, is arranged in a folded state in a gap between the underlying front sheet and the non-adhesive region of the reverse side of the overlying front sheet;
said disposal pouch is made of an impermeable sheet material so as to enable sealed enclosure of the paper diaper after use to confine the foul odors of waste to a minimum during disposal;
said disposal pouch is arranged in such a way as not to come loose from the paper diaper easily; and
in at least one location on said overlying front sheet an opening is provided for withdrawal of said disposal pouch stored inside.

8. A disposal-pouch-included paper diaper as set forth in claim 7, the disposal-pouch-included paper diaper being wherein said disposal pouch is characterized in that part of the pouch is adhered either to the diaper body, or to said front sheet.

9. A disposal-pouch-included paper diaper as set forth in claim 7, the disposal-pouch-included paper diaper characterized in that:
a perforation of a length that lets the contained disposal pouch be withdrawn is provided for the disposal-pouch withdrawal opening;
the front sheet is rectangular in form; and
the perforation is located along at least one of the longer edges, the shorter edges, or the midsection of the rectangular front sheet.

10. A disposal-pouch-included paper diaper as set forth in claim 7, the disposal-pouch-included paper diaper characterized in that:
a slit of a length that lets the contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
part of the slit for the disposal-pouch withdrawal opening is locked by sticky tape to prevent the disposal pouch from coming loose.

11. A disposal-pouch-included paper diaper as set forth in claim 7, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
the slit for the pouch withdrawal opening is fitted with openable/closable Velcro™ to prevent the disposal pouch from coming loose.

12. A disposal-pouch-included paper diaper as set forth in claim 7, the disposal-pouch-included paper diaper characterized in that:
a perforation is provided in said paper-diaper disposal pouch in the vicinity of the end thereof adhered to the diaper body or the front sheet, whereby
said disposal pouch may be used by tearing the pouch off by the perforation and taking it out.

13. A paper diaper with an attached disposal pouch, in which fitted to a paper diaper is a disposal pouch in order for the paper diaper after use to be hygienically disposed of, the paper diaper comprising:
a paper-diaper body section having a front panel and a rear panel;
a front-sheet section; and
a disposal pouch; the disposal-pouch-included paper diaper characterized in that to impart both convenience and aesthetic appeal:
said front-sheet section along a peripheral portion of its reverse side has an adhesive surface, on a midportion of its reverse side has a non-adhesive region not possessing an adhesive surface, and is adhered to a waist-portion outer surface of the paper diaper body;
said disposal pouch, of a thin material in order to minimize its thickness, is arranged in a folded state in a gap between the paper-diaper body outer surface and the non-adhesive region of the reverse side of said front-sheet section;
said disposal pouch is made of an impermeable sheet material so as to enable sealed enclosure of the paper diaper after use to confine the foul odors of waste to a minimum during disposal;
said disposal pouch is arranged in such a way as not to come loose from the paper diaper easily; and
in at least one location on said front sheet an opening is provided for withdrawal of said disposal pouch stored inside.

14. A disposal-pouch-included paper diaper as set forth in claim 13, the disposal-pouch-included paper diaper being wherein said disposal pouch is characterized in that part of the pouch is adhered either to the diaper body, or to said front sheet.

15. A disposal-pouch-included paper diaper as set forth in claim 13, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
part of the disposal-pouch withdrawal opening is locked by sticky tape.

16. A disposal-pouch-included paper diaper as set forth in claim 13, the disposal-pouch-included paper diaper characterized in that:
a perforation of a length that lets the folded and contained disposal pouch be withdrawn is provided for the disposal-pouch withdrawal opening;
the front sheet is rectangular in form; and
the perforation is located along at least one of the longer edges, the shorter edges, or the midsection of the rectangular front sheet.

17. A disposal-pouch-included paper diaper as set forth in claim 13, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
the slit for the pouch withdrawal opening is fitted with openable/closable Velcro™ to prevent the disposal pouch from coming loose.

18. A disposal-pouch-included paper diaper as set forth in claim 13, the disposal-pouch-included paper diaper characterized in that:
a perforation is provided in said paper-diaper disposal pouch in the vicinity of the end thereof adhered to the diaper body or the front sheet, whereby
said disposal pouch may be used by tearing the pouch off by the perforation and taking it out.

19. A paper diaper with an attached disposal pouch, in which fitted to a paper diaper is a disposal pouch in order for the paper diaper after use to be hygienically disposed of, the paper diaper comprising:
a paper-diaper body section having a front panel and a rear panel;
a front-sheet section; and
a disposal pouch; the disposal-pouch-included paper diaper characterized in that to impart both convenience and aesthetic appeal:
said front-sheet section is arranged layered in two plies on a waist-portion outer surface of the paper-diaper body section, with the underlying front sheet being adhered to the paper-diaper body outer surface, and with the overlying front sheet along a peripheral portion of its reverse side having an adhesive surface, on a midportion of its reverse side having a non-adhesive region not possessing an adhesive surface, and being adhered onto, so as to overlie, the underlying front sheet;
said disposal pouch, of a thin material in order to minimize its thickness, is arranged in a folded state in a gap between the underlying front sheet and the non-adhesive region of the reverse side of the overlying front sheet;
said disposal pouch is made of an impermeable sheet material so as to enable sealed enclosure of the paper diaper after use to confine the foul odors of waste to a minimum during disposal;
said disposal pouch is arranged in such a way as not to come loose from the paper diaper easily; and
in at least one location on said overlying front sheet an opening is provided for withdrawal of said disposal pouch stored inside.

20. A disposal-pouch-included paper diaper as set forth in claim 19, the disposal-pouch-included paper diaper being wherein said disposal pouch is characterized in that part of the pouch is adhered either to the diaper body, or to said front sheet.

21. A disposal-pouch-included paper diaper as set forth in claim 19, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
part of the disposal-pouch withdrawal opening is locked by sticky tape.

22. A disposal-pouch-included paper diaper as set forth in claim 19, the disposal-pouch-included paper diaper characterized in that:
a perforation of a length that lets the folded and contained disposal pouch be withdrawn is provided for the disposal-pouch withdrawal opening;
the front sheet is rectangular in form; and
the perforation is located along at least one of the longer edges, the shorter edges, or the midsection of the rectangular front sheet.

23. A disposal-pouch-included paper diaper as set forth in claim 19, the disposal-pouch-included paper diaper characterized in that:
a slit of a length that lets the folded and contained disposal pouch be withdrawn is provided for the disposal-pouch withdrawal opening;
the front sheet is rectangular in form;
the disposal-pouch withdrawal opening is located along at least one of the longer edges or the shorter edges of the rectangular front sheet; and
the slit for the pouch withdrawal opening is fitted with openable/closable Velcro™ to prevent the disposal pouch from coming loose.

24. A disposal-pouch-included paper diaper as set forth in claim 19, the disposal-pouch-included paper diaper characterized in that:
a perforation is provided in said paper-diaper disposal pouch in the vicinity of the end thereof adhered to the diaper body or the front sheet, whereby
said disposal pouch may be used by tearing the pouch off by the perforation and taking it out.
